(19) United States
(12) Patent Application Publication
(10) Pub. No.: US 2026/0211093 A1
(43) Pub. Date: Jul. 23, 2026

(54) A METHOD AND SYSTEM FOR ULTRASONIC FOCUSING COMPENSATION

(71) Applicant: XSONICO TECHNOLOGY LIMITED, Beijing (CN)

(72) Inventors: Zhicheng BAI, Kunming (CN); Daoxiang PENG, Beijing (CN); Zhaoyi HAO, Beijing (CN)

(73) Assignee: XSONICO TECHNOLOGY LIMITED, Beijing (CN)

(21) Appl. No.: 18/871,546

(22) PCT Filed: Mar. 21, 2024
(86) PCT No.: PCT/CN2024/082844

§ 371 Date: Apr. 9, 2026

(30) Foreign Application Priority Data
Oct. 14, 2023 (CN) 202311328545.0

(51) Int. Cl. G01S 7/52; A61N 7/02; G10K 11/26

(52) U.S. Cl. CPC G01S 7/5206; G10K 11/26; A61N 7/02

(57) ABSTRACT

The present disclosure provides a method and system for ultrasonic focusing compensation. Ultrasonic waves are attenuated in biological tissues, and ultrasonic sound velocities in different biological tissues are different, so that different ultrasonic intensities and phases can be formed when the ultrasonic waves are focused on different parts in the biological tissues, different influence areas are formed at different positions of the biological tissues, and accurate control technology of HIFU is not facilitated. According to the ultrasonic focusing compensation method provided by the present disclosure, the initial intensity, the emission time or the initial phase of each ultrasonic element is compensated by adjusting the output of the ultrasonic transducer, so that the problems in the prior art are solved. The same ultrasonic intensity and phase can still be kept when high-intensity ultrasound is focused at different parts, thereby effectively improving the accuracy of the technology of HIFU.

### FIELD OF THE PRESENT DISCLOSURE

The present disclosure relates to the field of High Intensity Focused Ultrasound, and particularly relates to a method and system for ultrasonic focusing compensation.

### BACKGROUND OF THE PRESENT DISCLOSURE

High Intensity Focused Ultrasound (HIFU) provides a non-invasive treatment method for ablating tumors precisely. Its basic principle involves highly concentrating ultrasonic energy at a focus, thereby producing transient high-temperature effects and cavitation effects that destroy targeted tissue lesions for surgical ablation. HIFU treatments do not cause external wounds nor damage normal tissues along the ultrasonic focusing path, HIFU has been used in the clinical treatment of benign and malignant tumors such as uterine fibroids, liver cancer, kidney cancer, and breast cancer.

During the process of ultrasonic focusing, as ultrasonic waves attenuate within biological tissues and the sound speed varies among different biological tissues, different ultrasonic intensities will be formed at the focus when focused on different parts of the biological tissues. Moreover, as ultrasonic waves (ultrasonic elements) in different directions pass through the different biological tissues, the phases of the ultrasonic elements at the focus may also be different. The current technology of HIFU mainly utilizes the high temperature effect of high intensity focused ultrasound, which in itself needs to gather excessive ultrasonic energy at the focus, and does not require precise control. Even if the ultrasonic waves are provided by driving the transducer as a whole, the ultrasonic intensity and phase deviation at the focus will not have a substantial impact. However, for working modes with low-overload focus energy, such as multi-focus HIFU and HIFU based on cavitation effect, the overall driving of the transducer cannot eliminate the sound intensity and phase deviation of each ultrasonic wave at the focus, which may result in the inability to ablate the lesion, thereby leading to surgical failure.

### SUMMARY OF THE PRESENT DISCLOSURE

In view of the problem existing in the background, an object of the present disclosure is to provide a method and system for ultrasonic focusing compensation, and the technical solution is as follows.

In a first aspect of the present disclosure, the present disclosure provides a method for ultrasonic focusing compensation. Generally speaking, this method is applied in the control process of HIFU equipment to improve the focusing effect of the ultrasonic waves at the focus. Specifically, the method comprises the following steps.

Step S1 determines the acoustic attenuation coefficients and penetration distances of each biological tissue along an ultrasonic focusing path.

Step S2 determines the ultrasonic intensities at the focus.

Step S3 compensates the initial intensities of ultrasonic waves by adjusting the output of an ultrasonic transducer, so that the ultrasonic intensities at the focus are consistent with the desired intensities.

Specifically, the step S1 calculates the acoustic attenuation coefficients and penetration distances of each biological tissue along the ultrasonic focusing path by establishing a computational model of the target biological tissue. This computational model is obtained based on a pre-established theoretical model and adjusted according to the age, weight and size of the target biological tissue. In a preferred embodiment, the step S1 obtains the acoustic attenuation coefficients and penetration distances of each biological tissue along the ultrasonic focusing path by performing MRI measurement on the biological tissue.

Specifically, the step S2 determines the ultrasonic intensity at a certain depth by the following formula (1):

$$I ( z ) = {I}_{0} {e}^{- 2 α \int {I}_{0} \hat{} zdz} . \qquad ( 1 )$$

I₀ represents the initial intensity of the ultrasonic wave, a represents the acoustic attenuation coefficient of the ultrasonic wave, z represents the depth reached by the ultrasonic wave, and I(z) represents the ultrasonic intensity at the depth.

Based on the ultrasound intensity at a certain depth obtained by the formula (1), in the step S3, the initial intensity of the ultrasound wave is compensated by adjusting the output of the ultrasonic transducer so that the compensated initial intensity meets the following formula (2):

$${I}_{0} = {I}_{target} / {e}^{- 2 α \int {I}_{0} \hat{} zdz} . \qquad ( 2 )$$

I_{target} is the ultrasonic intensity expected to be obtained at a target depth, that is, the expected ultrasonic intensity. Through the above-mentioned focusing compensation process, the ultrasonic intensities at the focus can be kept the same at different penetration distances, thereby achieving a good focusing control effect.

Preferably, considering that the ultrasonic wave may pass through a variety of biological tissues along its ultrasonic focusing path and the biological tissues passed by each ultrasonic wave may be different, the step S2 determines the ultrasonic intensity of the j-th ultrasonic element at the focus by the following formula (3):

$${I}_{j} ( z ) = {I}_{0} * {\prod }_{i = 1}^{n} {e}^{- 2 {α}_{ij} {z}_{ij}} . \qquad ( 3 )$$

Iⱼ(z) represents the ultrasonic intensity of the ultrasonic wave at depth z, I₀ represents the initial intensity of the ultrasonic wave, αᵢⱼ represents the acoustic attenuation coefficient of the j-th ultrasonic element passes through the i-th biological tissue, and zᵢⱼ represents the penetration distance that the j-th ultrasonic element penetrates the i-th biological tissue.

Based on the ultrasonic intensity at the focus obtained by the formula (3), in step S3, the initial intensity of the ultrasonic wave is compensated by adjusting the output of the ultrasonic transducer so that the compensated initial intensity meets the following formula (4):

$${I}_{0 j} = {I}_{target} / {\prod }_{i = 1}^{n} {e}^{- 2 {α}_{ij} {z}_{ij}} . \qquad ( 4 )$$

I₀ⱼ represents the actual initial intensity of the j-th ultrasonic element after being compensated, as the biological tissues passed through by each ultrasonic wave on its ultrasonic focusing path may be different, the initial intensities of each ultrasonic element need to be determined separately. Through the process of ultrasonic focusing compensation, the ultrasonic intensities at the focus can be kept the same at different penetration distances more accurately, thereby achieving a good focusing control effect.

Further preferably, taking into account that the different biological tissues passed through by each ultrasonic wave on its ultrasonic focusing path may also cause the phases of each ultrasonic element at the focus to deviate, the step S1 also determines the sound velocities of each biological tissue along the ultrasonic focusing path, the step S2 also determines the time required for each ultrasonic wave to reach the focus, and the step S3 also compensates for the start time of transmitting each ultrasonic wave by adjusting the output of the ultrasonic transducer, so that the phases of each ultrasonic wave at the focus are remained consistent.

Specifically, similar to the other parameters mentioned above, the step S1 can obtain the sound velocities of each biological tissue along the ultrasonic focusing path by establishing a computational model of the target biological tissue, or can obtain them by performing an ultrasonic measurement of B-mode on the biological tissues.

Specifically, the step S2 also determines the time required for each ultrasonic wave to reach the focus by the following formula (5):

$${T}_{j} = {\sum }_{i = 1}^{n} \frac{{z}_{ij}}{{v}_{ij}} . \qquad ( 5 )$$

Tⱼ represents the time required for the j-th ultrasonic element to reach the focus, vᵢⱼ represents the sound velocity of the j-th ultrasonic element passes through the i-th biological tissue, and zᵢⱼ represents the penetration distance that the j-th ultrasonic element penetrates the i-th biological tissue.

Based on the time required for each ultrasonic wave to reach the focus obtained by the formula (5), in the step S3, the start time of transmitting each ultrasonic element are compensated by adjusting the output of the ultrasonic transducer, so that the delay time of the start time of transmitting each ultrasonic element meet the following formula (6):

$$Δ {T}_{j} = {T}_{target} - {\sum }_{i = 1}^{n} \frac{{z}_{ij}}{{v}_{ij}} . \qquad ( 6 )$$

ΔTⱼ is the delay time of the start time of transmitting the j-th ultrasonic element, T_{target} is the preset time when each ultrasonic element is expected to focus on the focus, the time is not less than the maximum time required for each ultrasonic element to reach the focus.

In addition to compensating the start time of transmitting each ultrasonic wave, in an alternative solution, the step S3 can also compensate the initial phases of each ultrasonic wave when it is transmitted by adjusting the output of the ultrasonic transducer according to the following formula (7), so that the phases of each ultrasonic wave at the focus can also be kept consistent:

$$Δ {φ}_{j} = {φ}_{target} - {\sum }_{i = 1}^{n} \frac{ω {z}_{ij}}{{v}_{ij}} . \qquad ( 7 )$$

Δφⱼ is the compensation amount of the phase when the j-th ultrasonic element is transmitted, φ_{target} is the preset initial phase when each ultrasonic element is expected to focus on the focus, and ω is the frequency of the ultrasonic wave.

In a second aspect of the present disclosure, the present disclosure provides a system for ultrasonic focusing compensation, which comprises an ultrasonic transducer, a computer, and a controller, wherein the ultrasonic transducer is used to transmit ultrasonic waves, the computer is used to execute the aforementioned the method for ultrasonic focusing compensation to obtain a required compensation amount of the ultrasonic focusing compensation, and the controller is used to control the output of the ultrasonic transducer according to the required compensation amount of the ultrasonic focusing compensation.

According to the method and system for ultrasonic focusing compensation provided by the present disclosure, the present disclosure has the following beneficial effects:

The present disclosure can enable the ultrasonic intensities of the high-intensity ultrasonic waves within biological tissues at different depths remain consistent, thereby avoiding the varying influence ranges of HIFU when the depth of the focus changes, and effectively improving the focusing effect of the ultrasonic waves. Furthermore, the present disclosure can overcome the influence of different biological tissues on each ultrasonic focusing path in complex focusing scenarios, maintain the same phase of each ultrasonic element at the focus, achieve a good focusing effect, and make the control process of HIFU more precise.

The above introduces some of the technical effects achieved by the present disclosure. It should be understood that the summary of the present disclosure is provided to introduce a selection of concepts in simplified form, and that are further described below in the detailed description. The summary of the present disclosure is not intended to identify key features or essential features of the present disclosure, nor is it intended to limit the scope of the present disclosure.

### BRIEF DESCRIPTION OF THE FIGURES

The above and other objects, features and advantages of the present disclosure will become more apparent through a more detailed description of exemplary embodiments of the present disclosure in conjunction with the accompanying drawings, wherein like reference numerals generally represent like parts throughout the exemplary embodiments of the present disclosure.

FIG. 1 shows a focusing schematic diagram of HIFU in the prior art;

FIG. 2 is a focusing schematic diagram of HIFU in an embodiment of the present disclosure.

Reference numerals in figures are represented as follows: 1—ultrasonic transducer; 2—influence range of focused ultrasound.

### DETAILED DESCRIPTION

Embodiments of the present disclosure will be described in more detail below with reference to the accompanying drawings. Although embodiments of the present disclosure are shown in the drawings, it should be understood that the present disclosure can be embodied in various forms and should not be limited to the embodiments set forth herein. On the contrary, these embodiments are provided to make the present disclosure more thorough and complete, and to fully convey the scope of the present disclosure to those skilled in the art.

As used herein, the term “include” and variations thereof means open inclusion, ie, “including but not limited to”. Unless otherwise stated, the term “or” means “and/or”. The term “based on” means “based at least in part on”. The terms “one example embodiment” and “an embodiment” means “at least one example embodiment”. The term “another embodiment” means “at least one additional embodiment”. The terms “first”, “second”, etc. may refer to different or the same objects. The following may include additional explicit and implicit definitions.

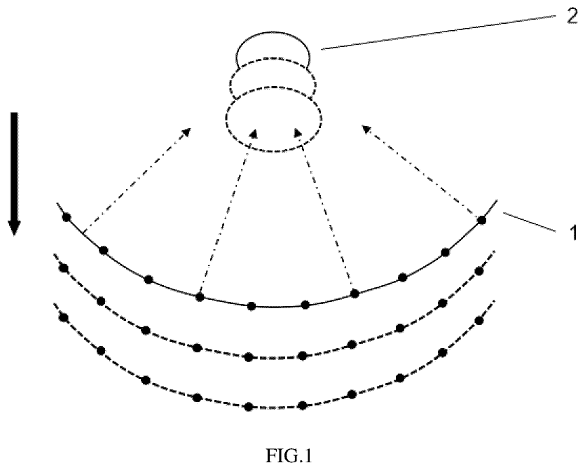

FIG. 1 shows a focusing schematic diagram of HIFU in the prior art. As shown in FIG. 1, as the focal depth gradually becomes shallower, the ultrasonic intensity at the focus gradually becomes stronger, causing the impact of HIFU to gradually increase. This phenomenon is not conducive to the precise control of the technology of HIFU.

According to the content disclosed in the present disclosure, this specific embodiment provides a preferred method for ultrasonic focusing compensation, which mainly includes the following steps.

The step S1 determines the acoustic attenuation coefficients, penetration distances and sound velocities of each biological tissue on an ultrasonic focusing path.

The step S2 determines the ultrasonic intensities at the focus and the time required for each ultrasonic wave to reach the focus.

The step S3 determines the initial ultrasonic intensities and the start time of transmitting each ultrasonic wave by adjusting the output of the ultrasonic transducer, so that the ultrasonic intensities of each ultrasonic wave at the focus are consistent with the desired intensities and the phases of each ultrasonic wave at the focus are remained consistent.

Specifically, the step S1 preferably obtains the acoustic attenuation coefficients, penetration distances, and sound velocities of each biological tissue on its ultrasonic focusing path by means of ultrasonic measurement of B-mode. In addition, these parameters can also be calculated by establishing a computational model, but it is necessary to establish a theoretical model of biological tissue in advance through experiments or reference to materials, and then adjust the theoretical model based on physiological data such as age, weight, size, etc. obtained from actual tests. Compared with the ultrasonic measurement of B-mode, the parameters obtained by the computational model may have certain deviations, but they can control costs while maintaining appropriate accuracy.

Based on the acoustic attenuation coefficients, penetration distances and sound velocities determined in the step S1, the step S2 can determine the ultrasonic intensities at the focus and the time required for each ultrasonic wave to reach the focus by the following formulas:

$${I}_{j} ( z ) = {I}_{0} * {\prod }_{i = 1}^{n} {e}^{- 2 {α}_{ij} {z}_{ij}} . \qquad ( 3 )$$

$${T}_{j} = {\sum }_{i = 1}^{n} \frac{{z}_{ij}}{{v}_{ij}} . \qquad ( 5 )$$

αᵢⱼ represents the acoustic attenuation coefficient of the j-th ultrasonic element passes through the i-th biological tissue, zᵢⱼ represents the penetration distance that the j-th ultrasonic element penetrates the i-th biological tissue, and vᵢⱼ represents the sound velocity of the j-th ultrasonic element passes through the i-th biological tissue. It should be understood that if the ultrasonic focusing paths of each ultrasonic wave focused to the focus pass through exactly the same biological tissue, such as muscle, fat or bone, then the Iⱼ(z) and Tⱼ of each ultrasonic wave are the same, but usually the actual situation does not conform to this ideal assumption, so a focusing compensation is required in the step S3.

Specifically, the step S3 compensates the initial intensities I₀ⱼ of each ultrasonic wave by the following formula, to ensure that the ultrasonic intensities of each ultrasonic wave at the focus are consistent:

$${I}_{0 j} = {I}_{target} / {\prod }_{i = 1}^{n} {e}^{- 2 {α}_{ij} {z}_{ij}} . \qquad ( 4 )$$

At the same time, the step S3 compensates the start time of transmitting each ultrasonic wave by the following formula, to ensure that the phases of each ultrasonic wave at the focus are consistent:

$$Δ {T}_{j} = {T}_{t a r g e t} - {\sum }_{i = 1}^{n} \frac{{z}_{ij}}{{v}_{ij}} . \qquad ( 6 )$$

This embodiment selects the maximum time Tₘₐₓ required for each ultrasonic element to reach the focus as T_{target}, and transmits the other ultrasonic waves by delaying the delay time ΔTⱼ to make each ultrasonic wave reach the focus at the same time.

In another specific implementation manner, the step S3 can also compensate the initial phases of each ultrasonic wave by the following formula, which can also ensure that the phases of each ultrasonic wave at the focus are consistent:

$$Δ {φ}_{j} = {φ}_{t a r g e t} - {\sum }_{i = 1}^{n} \frac{ω {z}_{i j}}{{v}_{i j}} . \qquad ( 7 )$$

Δφⱼ is the compensation amount of the phase when the j-th ultrasonic element is transmitted, φ_{target} is the preset initial phase when each ultrasonic element is expected to focus on the focus, and co is the frequency of the ultrasonic wave.

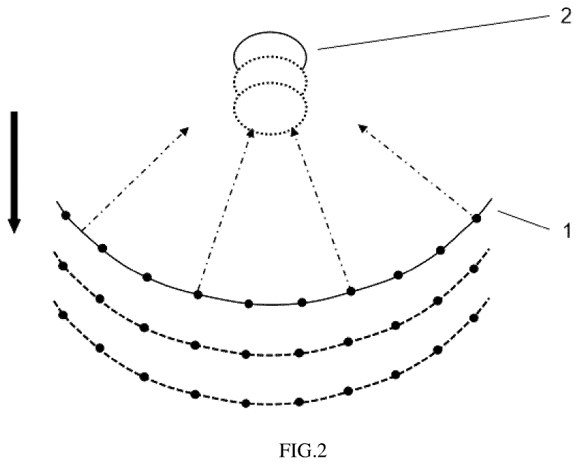

By using the method for ultrasonic focusing compensation provided in this specific embodiment, as shown in FIG. 2, as the focusing position of the ultrasonic transducer 1 changes in depth, the ultrasonic intensities at the focus are remained constant, and the phases of each ultrasonic element are remained consistent, so that the ultrasound focusing of HIFU is more precise, which is beneficial to the precise control of the technology of HIFU.

Various embodiments of the present disclosure have been described above. The above description is exemplary, not exhaustive, and is not limited to the disclosed embodiments. Many modifications and variations will be apparent to those of ordinary skill in the art without departing from the scope and spirit of the described embodiments. The terms used herein are selected to best explain the principles of the embodiments, practical applications, or improvements over prior art, or to enable others of ordinary skill in the art to understand the embodiments disclosed herein.

What is claimed is:

1. A method for ultrasonic focusing compensation, wherein comprising the following steps:
S1. determining the acoustic attenuation coefficients and penetration distances of biological tissues on an ultrasonic focusing path;
S2. determining the ultrasonic intensities of ultrasonic waves at the focus;
S3. by adjusting the output of an ultrasonic transducer, the initial intensities of the ultrasonic waves are compensated so that the ultrasonic intensities at the focus are consistent with the desired intensities;
the step S2 determines the ultrasonic intensity of the j-th ultrasonic element at the focus by the following formula:
$${I}_{j} ( z ) = {I}_{0} * {\prod }_{i = 1}^{n} {e}^{- 2 {α}_{ij} {z}_{ij}} ;$$
Iⱼ(z) represents the ultrasonic intensity of the ultrasonic wave at depth z, I₀ represents the initial intensity of the ultrasonic element, αᵢⱼ represents the acoustic attenuation coefficient of the j-th ultrasonic element passes through the i-th biological tissue, zᵢⱼ represents the penetration distance that the j-th ultrasonic element penetrates the i-th biological tissue;
the step S3 compensates for the initial intensities of the ultrasonic elements by adjusting the output of the ultrasonic transducer, so that the compensated initial intensities of each ultrasonic element meet the following formula:
$${I}_{0 j} = {I}_{t a r g e t} / {\prod }_{i = 1}^{n} {e}^{- 2 {α}_{ij} {z}_{ij}} ;$$
I₀ⱼ represents the actual initial intensity of the j-th ultrasonic element after being compensated, I_{target} is the ultrasonic intensity desired to be obtained at the focus.

2. The method for ultrasonic focusing compensation according to claim 1, wherein,
The step S1 calculates the acoustic attenuation coefficients and penetration distances of each biological tissue along the ultrasonic focusing path by establishing a computational model of the target biological tissue;
The computational model is obtained based on a pre-established theoretical model and adjusted according to the age, weight and size of the target biological tissue.

3. The method for ultrasonic focusing compensation according to claim 1, wherein,
The step S1 obtains the acoustic attenuation coefficients and penetration distances of each biological tissue along the ultrasonic focusing path by performing MRI measurement on the biological tissue and comparing the databases.

4. The method for ultrasonic focusing compensation according to claim 1, wherein,
The step S1 also determines the sound velocities of each biological tissue along the ultrasonic focusing path, the step S2 also determines the time required for each ultrasonic wave to reach the focus, the step S3 also compensates for the start time of transmitting each ultrasonic wave by adjusting the output of the ultrasonic transducer, so that the phases of the ultrasonic waves at the focus are remained consistent.

5. The method for ultrasonic focusing compensation according to claim 4, wherein,
the step S2 also determines the time required for each ultrasonic wave to reach the focus by the following formula:
$${T}_{j} = {\sum }_{i = 1}^{n} \frac{{z}_{ij}}{{v}_{ij}} ;$$
Tⱼ represents the time required for the j-th ultrasonic element to reach the focus, vᵢⱼ represents the sound velocity of the j-th ultrasonic element passes through the i-th biological tissue, zᵢⱼ represents the penetration distance that the j-th ultrasonic element penetrates the i-th biological tissue;
the step S3 compensates for the start time of transmitting each ultrasonic element by adjusting the output of the ultrasonic transducer, so that the delay time of the start time of transmitting each ultrasonic element meet the following formula:
$$Δ {T}_{j} = {T}_{t a r g e t} - {\sum }_{i = 1}^{n} \frac{{z}_{ij}}{{v}_{ij}} ;$$
ΔTⱼ is the delay time of the start time of transmitting the j-th ultrasonic element, T_{target} is the time each ultrasonic element is desired to be obtained at the focus, the time is not less than the maximum time required for each ultrasonic element to reach the focus.

6. The method for ultrasonic focusing compensation according to claim 1, wherein,
The step S1 also determines the sound velocities of each biological tissue along the ultrasonic focusing path, the step S2 also determines the time required for each ultrasonic element to reach the focus, the step S3 also compensates for the initial phases of each ultrasonic element by adjusting the output of the ultrasonic transducer, so that the phases of each ultrasonic element at the focus are remained consistent.

7. The method for ultrasonic focusing compensation according to claim 6, wherein,
the step S2 determines the time required for each ultrasonic element to reach the focus by the following formula:
$${T}_{j} = {\sum }_{i = 1}^{n} \frac{{z}_{ij}}{{v}_{ij}} ;$$
Tⱼ represents the time required for the j-th ultrasonic element to reach the focus, vᵢⱼ represents the sound velocity of the j-th ultrasonic element passes through the i-th biological tissue, zᵢⱼ represents the penetration distance that the j-th ultrasonic element penetrates the i-th biological tissue;
by adjusting the output of the ultrasonic transducer, the step S3 compensates for the initial phases of each ultrasonic element by the following formula:
$$Δ {φ}_{j} = {φ}_{t a r g e t} - {\sum }_{i = 1}^{n} \frac{ω {z}_{i j}}{{v}_{i j}} ;$$
Δφⱼ is the compensation amount of phase when the j-th ultrasonic element is transmitted, φ_{target} is the initial phase when each ultrasonic element is expected to focus on the focus, ω is the frequency of the ultrasonic wave.

8. A system for ultrasonic focusing compensation, wherein, comprising an ultrasonic transducer, a computer, and a controller;
the ultrasonic transducer is used to transmit ultrasonic waves;
the computer is used to execute the method for ultrasonic focusing compensation according to any one of claims 1 to 7;
the controller is used to control the output of the ultrasonic transducer.
